(19) United States
(12) Patent Application Publication
(10) Pub. No.: US 2020/0338310 A1
(43) Pub. Date: Oct. 29, 2020

(54) SECURE PERIPHERALLY INSERTED CENTRAL CATHETER (PICC)

(71) Applicant: SECURE PICC PARTNERS, LLC, Loudonville, NY (US)

(72) Inventors: R. Maxwell Alley, Loudonville, NY (US); David Bruce, Altamont, NY (US); Kyrsten Maeve Carlson, Lebanon, NH (US); Mary Clare Crochiere, Wilbraham, MA (US); Jacob Kyle Ferreira, Mapleville, RI (US); Nisha Rajamohan, Waterford, CT (US); Ayla Yagan, Syracuse, NY (US)

(21) Appl. No.: 16/856,263

(22) Filed: Apr. 23, 2020

Related U.S. Application Data
(60) Provisional application No. 62/920,389, filed on Apr. 29, 2019.

(51) Int. Cl. A61M 25/02; A61M 25/00; A61M 39/10

(52) U.S. Cl. CPC A61M 25/02; A61M 25/0045; A61M 2039/1077; A61M 2025/0213; A61M 2025/0048; A61M 39/10

(57) ABSTRACT

Various implementations include peripherally inserted central catheters (PICCs) and methods for using PICCs. In a particular implementation, a PICC includes: a lumen having an innermost layer, at least one middle layer, an outermost layer, and a proximal end for inserting into a vessel, where the lumen is puncture resistant; a luer locking mechanism coupled to a distal end of the lumen, the luer locking mechanism having at least one injection port for mating with an external injectable medical device; and a housing unit at least partially surrounding the luer locking mechanism and preventing access to the at least one injection port.

### PRIORITY CLAIM

This application claims priority to U.S. Provisional Patent Application No. 62/920,389 (“Secure Peripherally Inserted Central Catheter”, filed on Apr. 29, 2019), which is hereby incorporated by reference in its entirety.

### TECHNICAL FIELD

This disclosure generally relates to peripherally inserted central catheters (also called, PICCs or PICC lines) and securing such PICCs. More particularly, the disclosure relates to protecting PICCs from undesired access and/or tampering.

### BACKGROUND

Peripherally-inserted central catheters, or PICCs, can be used to treat patients that require prolonged recurrent intravenous (IV) treatment. PICCs reduce the number of needle punctures a patient endures from IV lines. However, conventional PICCs can be subject to misuse and/or tampering, which can be particularly hazardous for patients that are also IV drug users. Preventing such misuse is challenging.

### SUMMARY

All examples and features mentioned below can be combined in any technically possible way.

Various implementations include PICCs and methods for using PICCs. Certain implementations include a PICC with a puncture-resistant and/or tamper-resistant lumen and a luer lock mechanism for preventing access to an injection port.

In particular aspects, a peripherally inserted central catheter (PICC) includes: a lumen having an innermost layer, at least one middle layer, an outermost layer, and a proximal end for inserting into a vessel, where the lumen is puncture resistant; a luer locking mechanism coupled to a distal end of the lumen, the luer locking mechanism having at least one injection port for mating with an external injectable medical device; and a housing unit at least partially surrounding the luer locking mechanism and preventing access to the at least one injection port.

In additional particular aspects, a method includes: inserting a peripherally inserted central catheter (PICC) into a patient, the inserting including: coupling a luer locking mechanism to a lumen, the luer locking mechanism having an injection port; coupling a housing unit to the luer locking mechanism; inserting the lumen into a designated patient area; and securing the housing unit to the luer locking mechanism to inhibit access to the injection port.

Implementations may include one of the following features, or any combination thereof.

In some cases, the innermost layer includes at least one of polyurethane or silicone; the middle layer includes a metal, a polymer, a synthetic fiber or a composite; and the outer layer includes polyurethane or silicone.

In some cases, the luer locking mechanism has a coupler for matingly engaging a coupler at the distal end of the lumen.

In particular cases, the luer locking mechanism engages with the distal end of the lumen via a set of interlocking threads, where the set of interlocking threads include a length and pitch that deviate from a reference thread by approximately ten percent.

In some cases, the luer locking mechanism has an entrance angle that deviates from a reference injection angle for a medical device port by at least approximately ten percent.

In some cases, the housing unit is selectively coupleable to an actuator for at least one of opening or closing the housing unit around the luer locking mechanism.

In some cases, the housing unit includes a first section and a second section, where the first section and the second section are matingly shaped, and when the first section is coupled with the second section the housing unit encloses the injection port.

In particular implementations, when the first section is coupled with the second section, the housing unit prevents access to the injection port.

In particular implementations, the housing unit has a means for selectively coupling the first section with the second section and where the selective coupling is performed with an external tool.

In some cases, the housing unit has a slot that is sized to receive the luer locking mechanism.

In some cases, the housing unit has a base and set of tabs extending from the base, the tabs including a set of interlocking threads, where the set of interlocking threads have a length and a pitch that deviate from a reference thread by approximately ten percent.

In some cases, the housing unit includes a base and a stem, where the stem is coupleable to the luer locking mechanism.

In particular implementations, the PICC includes a thread pairing having at least one threaded hole and at least one non-threaded hole, where in a held position the stem is retained in the non-threaded hole.

In particular implementations, the threaded hole of the thread pairing is selectively coupleable to an external tool for at least one of engaging or disengaging the stem of the housing unit.

In some cases, the inserting further includes: securing the housing unit to the luer locking mechanism using an authorized key to lock the housing unit.

In particular implementations, the inserting further includes: unlocking the housing unit using the authorized key.

In some cases, the inserting further includes: coupling a thread pairing to the housing unit, where the thread pairing holds the housing unit to secure the housing unit around the luer locking mechanism.

In particular implementations, the inserting further includes: using the thread pairing to inhibit access to the injection port by securing the housing unit around the luer locking mechanism.

In some cases, the inserting further includes: removing the inserted PICC from a patient, the removing including disengaging the luer locking mechanism from the patient and guiding the lumen out of the patient.

Two or more features described in this disclosure, including those described in this summary section, may be combined to form implementations not specifically described herein.

The details of one or more implementations are set forth in the accompanying drawings and the description below. Other features, objects and benefits will be apparent from the description and drawings, and from the claims.

### BRIEF DESCRIPTION OF THE DRAWINGS

FIG. 1 depicts a front view of a three layer lining of a lumen for a peripherally inserted central catheter (PICC) according to various implementations.

FIG. 2 depicts a side view of the three layer lining of the lumen of FIG. 1.

FIG. 3 depicts a top view of the three layer lining of the lumen of FIG. 1.

FIG. 4 depicts an isometric view of the three layer lining of the lumen of FIG. 1.

FIG. 5 depicts a front view of an injection port according to various implementations.

FIG. 6 is a side view of the injection port of FIG. 5, in which the threads are now visible according to various implementations.

FIG. 7 is a top view of the injection port of FIGS. 5 and 6.

FIG. 8 is an isometric view of the injection port of FIGS. 5-7, illustrating a luer locking mechanism according to various implementations.

FIG. 9 is a front view of a portion of a luer locking mechanism according to various implementations.

FIG. 10 is a side view of the portion of the luer locking mechanism of FIG. 9.

FIG. 11 is a top view of the portion of the luer locking mechanism of FIGS. 9 and 10.

FIG. 12 is an isometric view of the portion of the luer locking mechanism of FIGS. 9-11.

FIG. 13 is a front view of a locked luer locking mechanism according to various implementations.

FIG. 14 is a side view of the luer locking mechanism of FIG. 13.

FIG. 15 is a top view of a portion of the luer locking mechanism of FIGS. 13 and 14.

FIG. 16 is an isometric view of the luer locking mechanism according to various implementations.

FIG. 17 depicts the front expanded view of a luer locking mechanism according to various implementations.

FIG. 18 is a side view of the luer locking mechanism in FIG. 17.

FIG. 19 is a top view of a portion of a luer locking mechanism according to various implementations.

FIG. 20 is an isometric expanded view of a luer locking mechanism according to various implementations.

FIG. 21 is a front view of a housing unit according to various implementations.

FIG. 22 is a side view of the housing unit of FIG. 21.

FIG. 23 is a top view of the housing unit of FIGS. 21 and 22.

FIG. 24 is an isometric view of the housing unit in FIGS. 21-23.

FIG. 25 is a front view of a thread pairing according to various implementations.

FIG. 26 is a side view of the thread pairing of FIG. 25.

FIG. 27 is a top view of the thread pairing of FIGS. 25 and 26.

FIG. 28 is an isometric view of the thread pairing in FIGS. 25-27.

FIG. 29 is a front view a housing unit and thread pairing assembly according to various implementations.

FIG. 30 is a side view of the housing unit and thread pairing assembly of FIG. 29.

FIG. 31 is a top view of the housing unit and thread pairing assembly of FIGS. 29 and 30.

FIG. 32 is an isometric view of the housing unit and thread pairing assembly in FIGS. 29-31.

FIG. 33 is an expanded front view of a housing unit and tulip assembly according to various implementations.

FIG. 34 is a side view of the housing unit and tulip assembly in FIG. 33.

FIG. 35 is an isometric view of the housing unit and tulip assembly in FIGS. 33 and 34.

FIG. 36 illustrates an example configuration of a housing including a solenoid-based locking mechanism according to various implementations.

FIG. 37 illustrates an example configuration of a housing including a solenoid-based locking mechanism according to various additional implementations.

It is noted that the drawings of the various implementations are not necessarily to scale. The drawings are intended to depict only typical aspects of the disclosure, and therefore should not be considered as limiting the scope of the implementations. In the drawings, like numbering represents like elements between the drawings.

### DETAILED DESCRIPTION

This disclosure is based, at least in part, on the realization that a peripherally inserted central catheter (PICC) can be implemented with features to protect against unauthorized use and/or tampering. For example, a PICC with a tamper-resistant (e.g., puncture-resistant) lumen and a housing unit can provide protection against unauthorized use.

Commonly labeled components in the FIGURES are considered to be substantially equivalent components for the purposes of illustration, and redundant discussion of those components is omitted for clarity.

As described herein, PICCs are often used in cases where intravenous treatment is needed on a recurring or prolonged basis. A PICC includes a hollow tube that inserts into a peripheral vein in the body (e.g., arm) and feeds through the vein until it reaches the heart. The tube inside the arm connects to a port on the outside surface of the arm where medication (e.g., antibiotics, saline, chemotherapy, liquid nutrition or another drug with a pH level between roughly 5 and 9) can be injected directly into the bloodstream.

However, as noted herein, PICCs are prone to misuse, particularly by IV drug users (IVDU). A PICC provides the IVDU with access to continue use of illicit drugs. Having an unsecure PICC can also pose public health problems, as unrestricted access can lead to non-sterile tampering with the device, and unwanted bacteria growth and possible infection. The conventional PICC fails to effectively limit access to medical professionals (e.g., physicians and nurses).

One conventional approach to prevent PICC misuse is to hold patients suspected of being an IVDU as in-patient during their treatment. While this approach allows the medical professional(s) to monitor the PICC, it significantly increases the costs of treatment and reduces the available space in the medical facility for treating other patients. While some conventional medical devices aim to allow medical professionals to “lock” the PICC, these devices are merely tamper evident, meaning that they indicate misuse but do not effectively restrict the IVDU from access to the PICC.

In contrast to conventional devices, various implementations include a secure PICC that prevents unauthorized access and/or misuse. The secure PICC includes both a puncture-resistant lumen and a lockable housing for preventing access from unauthorized personnel.

In particular cases, a PICC is disclosed that includes: a lumen; a luer locking mechanism coupled to a distal end of the lumen; and a housing unit at least partially surrounding the luer locking mechanism. The lumen has multiple layers for preventing access to a core conduit that runs therethrough. The luer locking mechanism includes one or more injection ports for accessing the core conduit in the lumen, enabling an external medical device to deliver a fluid to a patient via the lumen. When closed/locked, the housing unit prevents access to the injection port. Components of the PICC (e.g., lumen, luer locking mechanism, housing, etc.) are disclosed and described in separate sections to provide detailed descriptions of their features and functions. However, as noted herein, these components can work in concert to provide the tamper-resistant features of the PICCs disclosed according to various implementations.

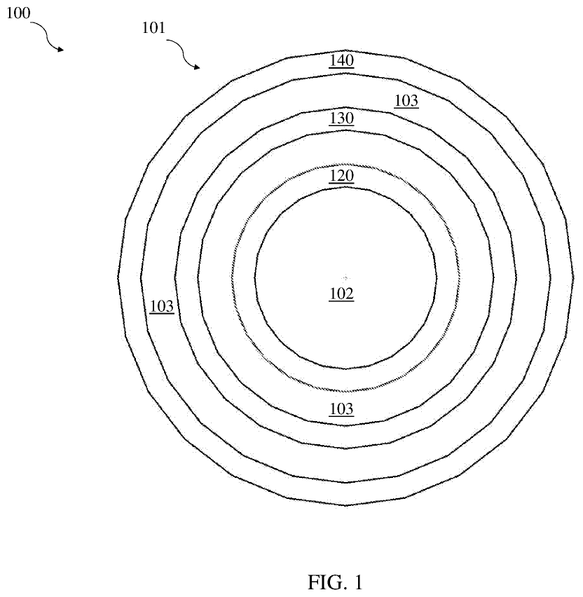

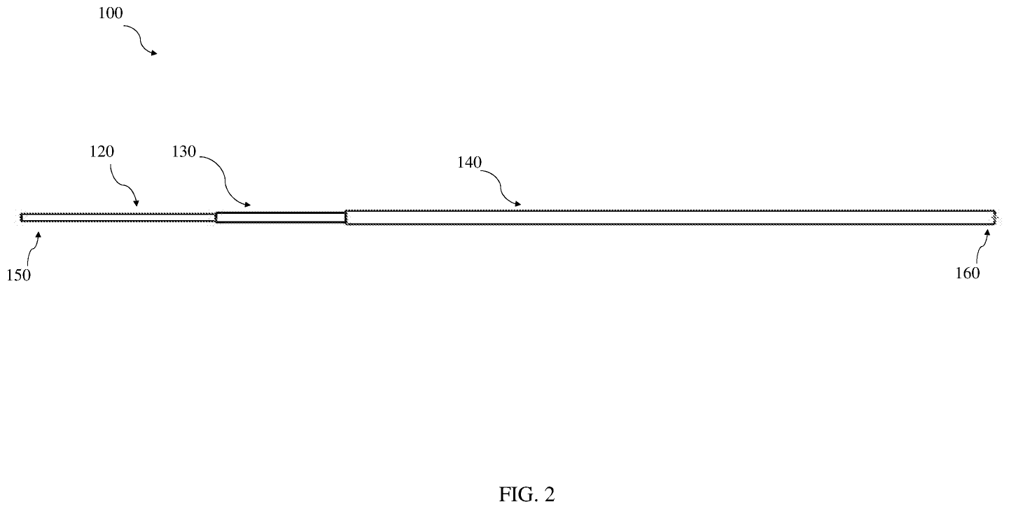

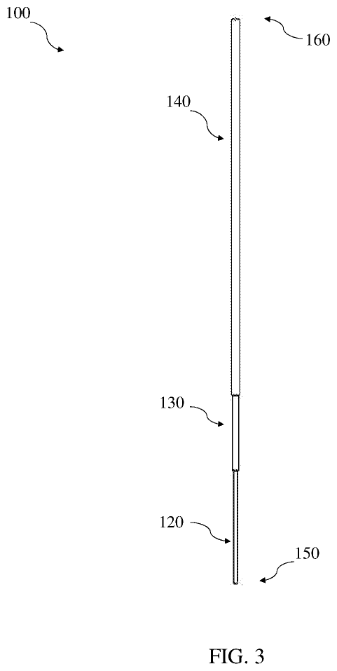

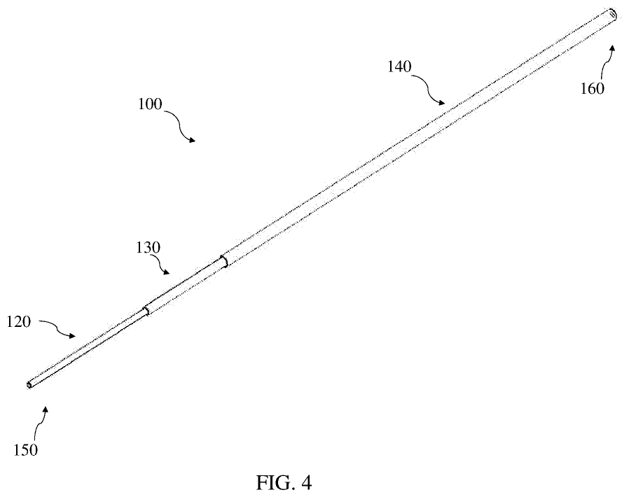

FIG. 1 illustrates a front view of a lumen 100 according to various implementations. FIG. 2 illustrates a side view of lumen 100, FIG. 3 illustrates a top view of lumen 100, and FIG. 4 illustrates an isometric view of lumen 100. These FIGURES are referred to simultaneously. In some cases, as shown in FIG. 1, the lumen 100 includes multiple layers 101 surrounding a conduit (tube) 102 for delivering a medical fluid such as antibiotics, saline, chemotherapy, liquid nutrition or another drug (e.g., with a pH level between approximately 5 and approximately 9). In particular cases, the layers 101 include: an innermost layer 120, at least one middle layer 130, and an outermost layer 140. Layers 101 may each be separated by an adhesive or bonding agent (referred to as “adhesive”) 103. In some embodiments, two or more layers 101 are fit together and fused at proximal end 150 and distal end 160. In some other embodiments, layers 101 are separated by a nearly-negligible amount of air. In some cases, the innermost layer 120 and outermost layer 140 include polyurethane or silicone. In particular implementations, the middle layer 130 is puncture resistant to prevent unauthorized access through the lumen 100, e.g., radially through the lumen 100 relative to the primary axis of medication delivery. In certain cases, the middle layer includes one or more of: a metal, a polymer, a composite, or a synthetic fiber (e.g., Kevlar or aramid). That is, the middle layer 130 is formed of a distinct material from the innermost layer 120 and the outermost layer 140. According to various implementations the middle layer 130 is formed of a material that is harder than a hypodermic medical needle, and in some cases, is puncture resistance up to a local applied force of approximately 10 Newton (N). In various embodiments, the middle layer 130 includes a plurality of distinct layers, e.g., two or more metal layers that are joined, or otherwise in contact with one another. The innermost layer 120 and outermost layer 140 are materially compatible to allow placement of the lumen 100 in a patient. That is, in various implementations, the innermost layer 120 and outermost layer 140 are formed of medical-grade material. As shown in FIGS. 2-4, the lumen 100 has a proximal end 150, and a distal end 160 opposite the proximal end 150. In practice, a medical professional inserts the proximal end 150 of the lumen 100 into a patient (e.g., an artery, capillary, vein, vessel, cavity) for delivering a medical fluid. In various implementations, the innermost layer 120, middle layer(s) 130 and outermost layer 140 have distinct lengths (e.g., axially). In various implementations, innermost layer 120, middle layer(s) 130 and outermost layer 140 may end concurrently, all with relatively equal length. However, in other implementations, the middle layer 130 only extends for a portion of the length of the lumen 100 as measured from the distal end 160. In these implementations, the middle layer 130 only extends from the distal end 160 of the lumen 100 by approximately several centimeters, e.g., to restrict tampering with the lumen 100 while inserted in the peripheral portion of the user's vein. As such, beyond this initial section near the distal end 160, the remaining portion of the lumen 100 has only two layers: the innermost layer 120 and the outermost layer 140. This section of the lumen 100 without the middle layer 130 may be more flexible than the section near the distal end 160, which can be beneficial for insertion and/or removal from the patient.

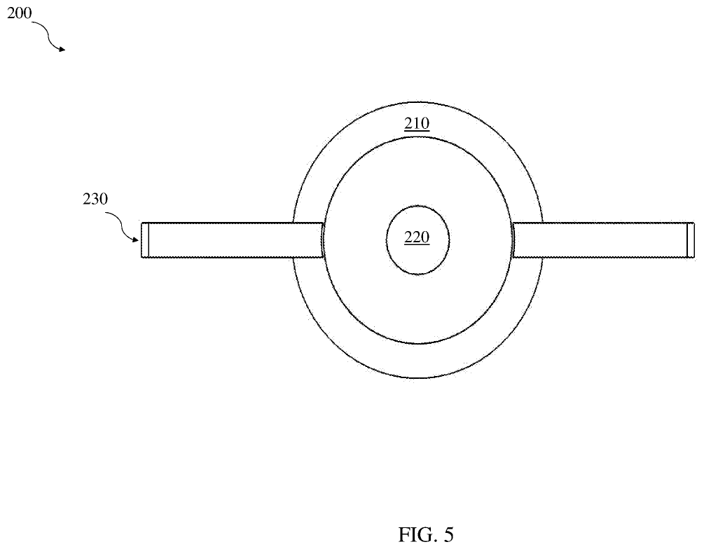

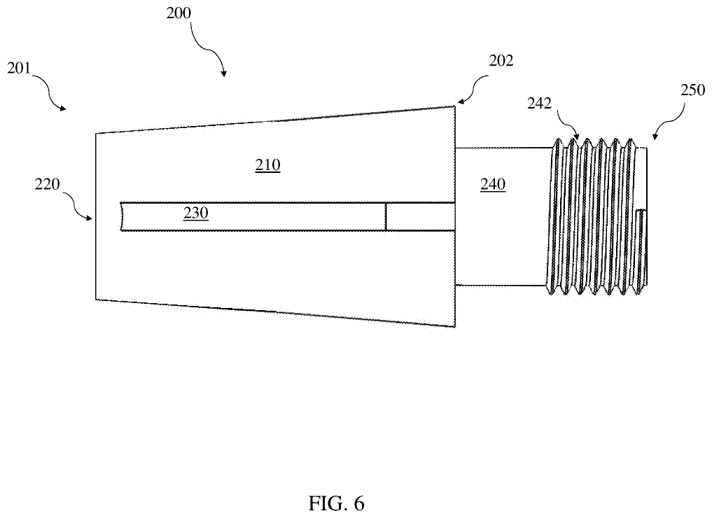

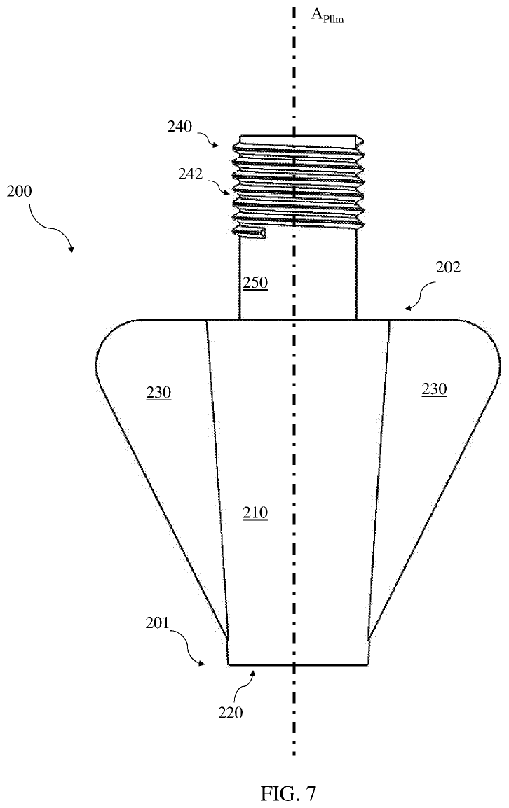

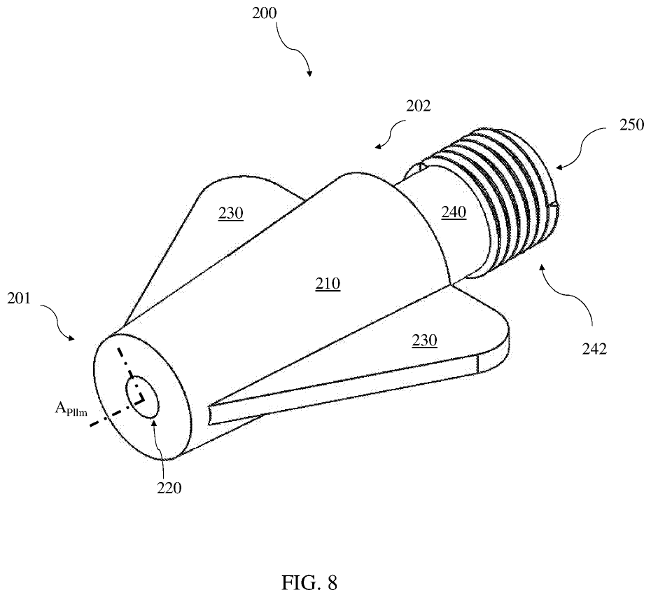

FIGS. 5-8 illustrate an example luer locking mechanism 200 for coupling with the distal end 160 of the lumen 100 according to various implementations. FIG. 5 illustrates an end view, FIG. 6 illustrates a side view, FIG. 7 illustrates a top view, and FIG. 8 illustrates an isometric view, respectively, of the luer locking mechanism 200. In various implementations, the distal end 160 of the lumen 100 (FIG. 2) is selectively coupleable to the luer locking mechanism 200 by a coupler 240 (FIGS. 6-8), which can include one or more of: interlocking threads, clips, pins, force-fitting members, adhesive, etc. That is, the luer locking mechanism 200 is selectively removable from the lumen 100 via the coupler 240.

In various implementations, the luer locking mechanism 200 has a main body 210 including a first (or, distal) end 201 and a second, opposite (or, proximal) end 202. In certain cases, the luer locking mechanism 200 tapers from the proximal end 202 to the distal end 201. The main body 210 may be made of any medical grade material. The main body 210 has a central channel for allowing medical fluid to pass therethrough. In various implementations, the luer locking mechanism 200 includes an injection port 220. The main body 210 separates the injection port 220 from the coupler 240. In particular cases, the injection port 220 is sized to matingly attach with an external injectable medical device to administer medical fluids to the patient through the lumen 100 (FIG. 2). For example, the injection port 220 can be sized to mate with a syringe or other fluid injection device. In particular cases, the injection port 220 is positioned such that a medical device (e.g., syringe) can be inserted to create an air-tight seal for administration of medical fluid. In some embodiments (not shown), the luer locking mechanism 200 has more than one injection port 220. Multiple injection ports 220 allow for multiple medical fluids to be administered in tandem or in conjunction. In some embodiments, the injection port 220 has a seal for mating with the external medical device.

As shown in FIGS. 5-8, in some cases, the luer locking mechanism 200 includes a set of arms 230. In various implementations, the set of arms 230 connect to (e.g., extend from) the main body 210, and in particular cases, the set of arms 230 are integral with the main body 210. The set of arms 230 are positioned to enable a user to actuate the coupler for engaging and/or disengaging the luer locking mechanism 200 from the lumen 100. In various implementations, the set of arms 230 extend radially from the main body 210 (relative to the primary axis (A_{Pllm}) of the luer locking mechanism 200). In particular cases, the arms 230 are capable of withstanding an outside (e.g., rotational) force to engage/disengage the luer locking mechanism 200 with the lumen 100, or other external medical devices. As shown, the luer locking mechanism 200 has a second end 250, e.g., for coupling with the lumen 100 and enabling medical fluid to pass therethrough. The main body 210 separates the second end 250 and the injection port 220. As shown, the second end 250 may include the coupler 240.

As can be seen in the example depiction of the luer locking mechanism 200 in FIG. 8, the coupler 240 can include a set of threads 242 for coupling (e.g., interlocking) with a complementary pair of threads in the lumen 100. In particular cases, the set of (interlocking) threads 242 have a diameter and pitch that deviate from a reference thread by approximately twenty percent. That is, the threads 242 can deviate from a reference (or standard) thread, and in some cases, include M3.5×0.6 threads that have a diameter size of 3.5 millimeters and 0.6 millimeter thread pitch. Threads 242 have an entrance angle of five degrees in some cases, and in particular cases, deviate from a reference entrance angle by approximately twenty percent. In some embodiments, the threads 242 are oriented counterclockwise, which is opposite reference (or standard) clockwise-oriented threads in medical devices. These deviations from reference or standard threads prevent common tools from coupling to the threads, e.g., mitigating tampering.

Further in the example depiction of the luer locking mechanism 200, in particular as shown in FIG. 8, the injection port 220 can include an entrance angle that deviates from a reference injection angle for a medical device port. In some cases, the injection port has an entrance angel that deviates from the reference injection angle (e.g., standard 3.44 degree angle measured relative to the normal angle from the face of port 220) by at least approximately twenty percent. This orientation can limit the types of injection devices that effectively pair with the port 220, e.g., mitigating tampering and/or misuse. The non-standard entrance angle of the port 220 helps prevent use by non-compatible devices (e.g., syringes). In various implementations, only compatible syringes can be used to access the port 220. These compatible syringes can be specific to this port entrance angle, and reserved, for example, for professional distribution and use.

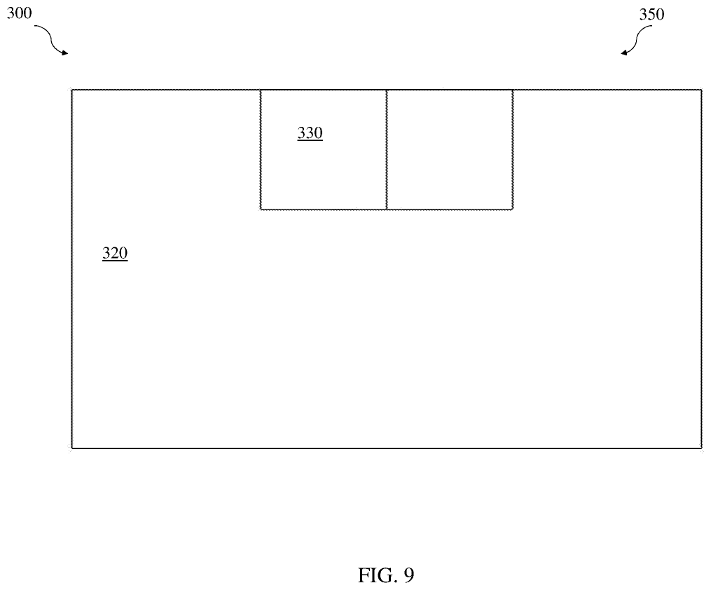

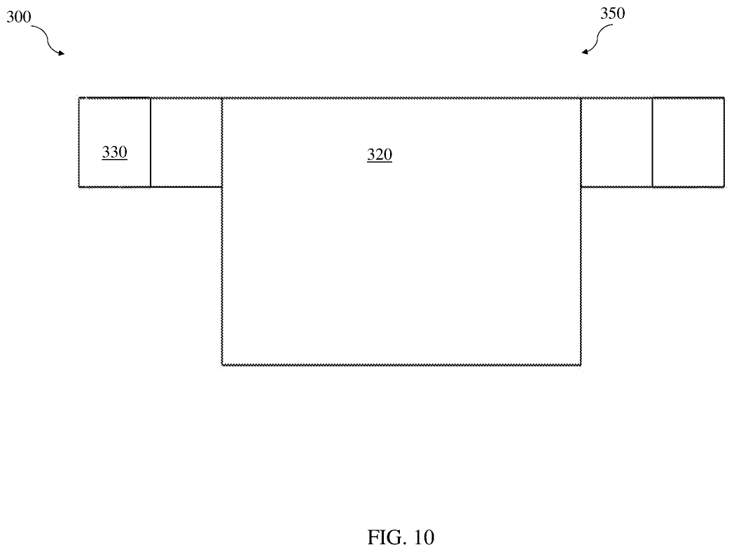

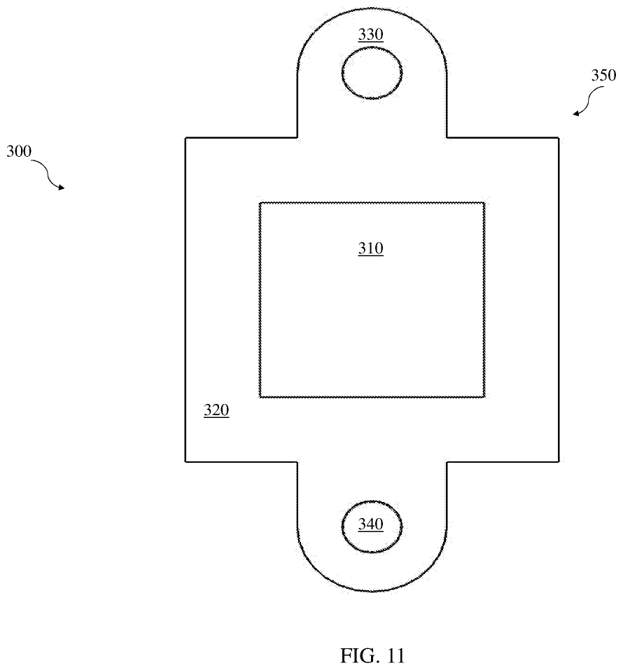

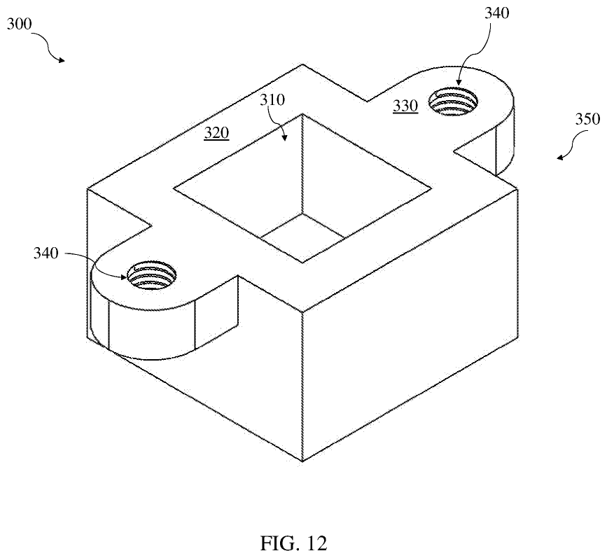

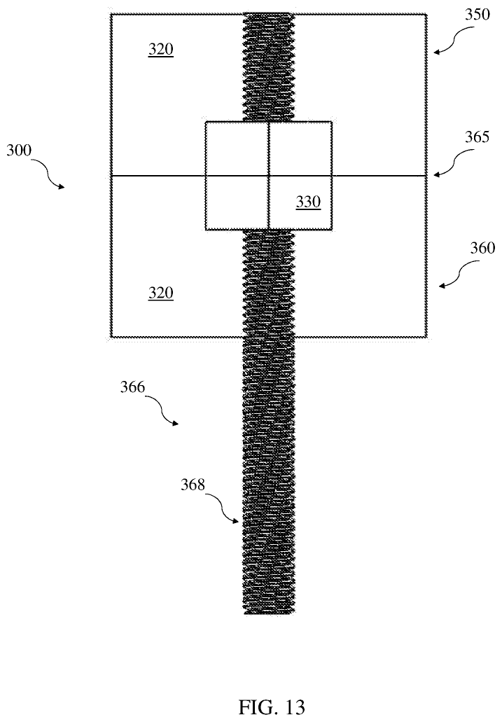

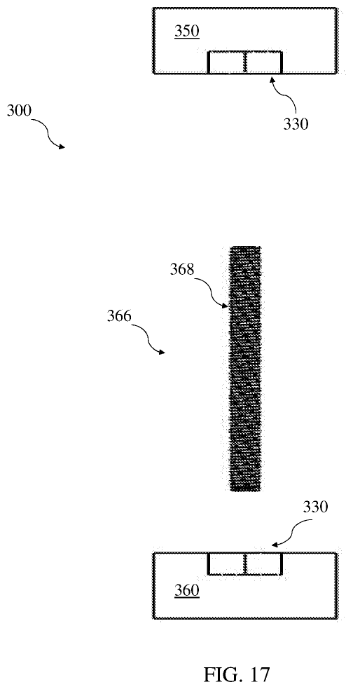

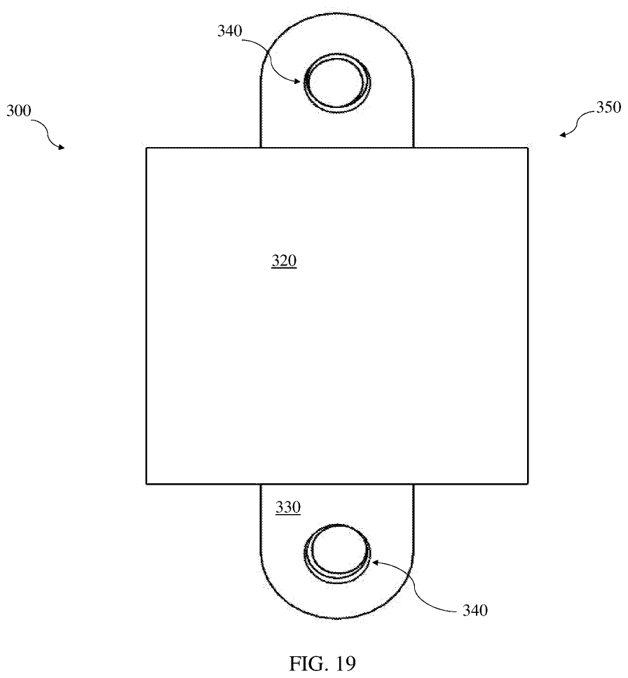

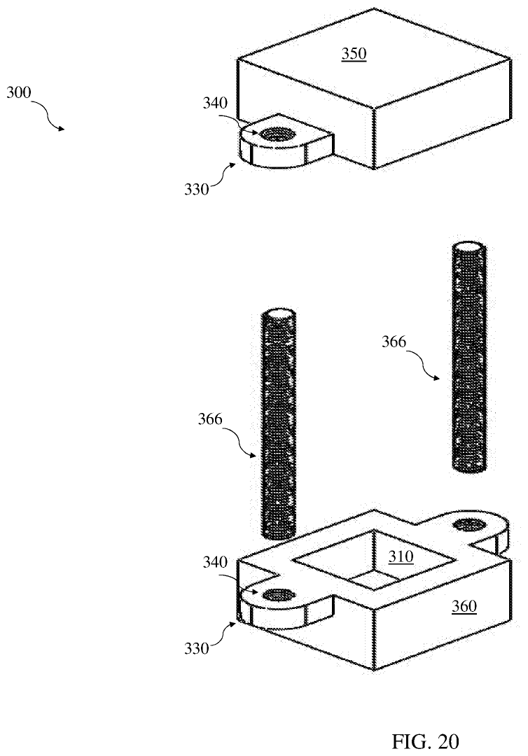

FIG. 9 illustrates a front view of a portion of a housing unit 300 according to various implementations. FIG. 10 illustrates a side view of a portion of the housing unit 300, FIG. 11 illustrates a top view of a portion of the housing unit 300, and FIG. 12 illustrates an isometric view of a portion of the housing unit 300. FIGS. 9-12 illustrate only a first section 350 of the housing unit 300, which is further described in complete form with reference to FIGS. 13-20. The housing unit 300 may take any shape capable of covering the injection port 220 (FIG. 8). That is, in various implementations, the housing unit 300 is sized and/or shaped to couple with the luer locking mechanism 200 and prevent access to the injection port 220 (FIG. 8). In various particular implementations, the housing unit 300 is sized to at least partially surround the luer locking mechanism 200 (FIG. 8) to prevent unauthorized access to the injection port 220. In certain cases, e.g., as shown in FIGS. 11 and 12, the housing unit 300 has a slot 310 that is sized to receive the luer locking mechanism 200, e.g., when the luer locking mechanism 200 is coupled with the lumen 100. In various implementations, the luer locking mechanism 200 may fit snuggly or loosely in the housing unit 300.

In one example depiction of the housing unit 300 in FIGS. 9-12, that housing unit 300 includes a base 320 and a set of tabs (e.g., extensions or “wings”) 330 extending from the base 320. The tabs 330 may each include a set of internal threads 340 for interlocking with a complementary component such as a connector. In certain cases, the internal threads 340 have a diameter and pitch that deviate from a reference thread by approximately twenty percent, and can include threads of M3.5×0.6, having a diameter of 3.5 millimeters and 0.6 millimeter thread pitch. The threads 340 may also, or alternatively, have an entrance angle of five degrees, or deviate from a reference entrance angle by approximately twenty percent. In another embodiment, the threads are oriented counterclockwise relative to the insertion axis.

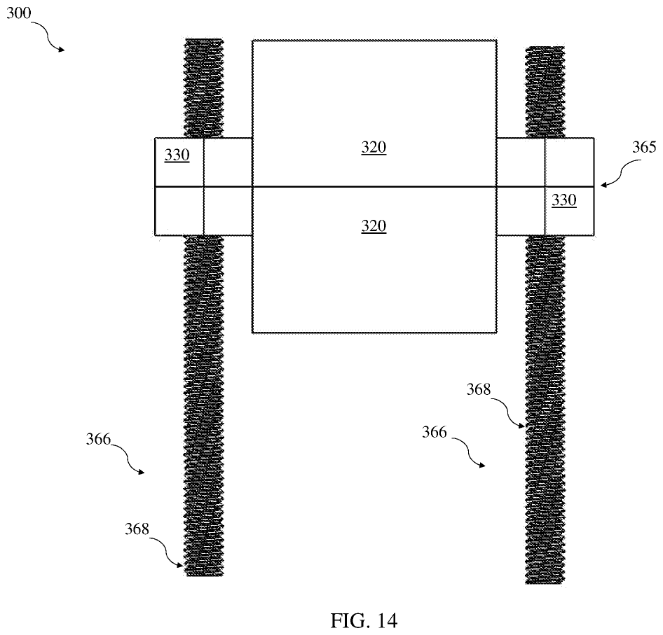

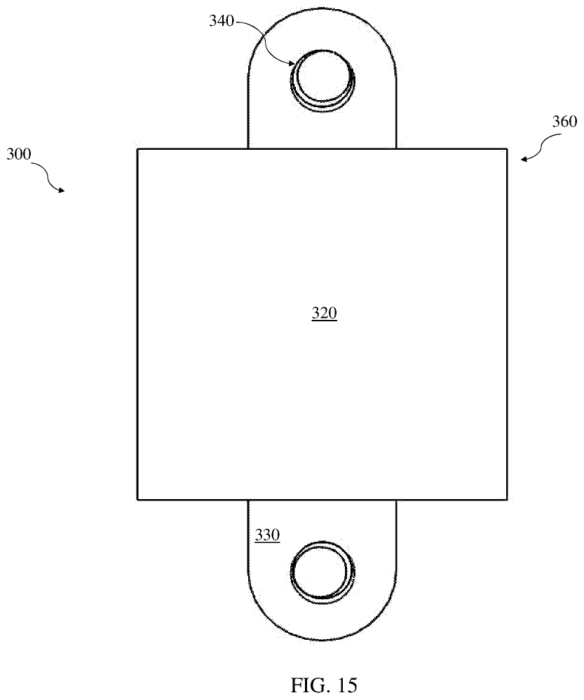

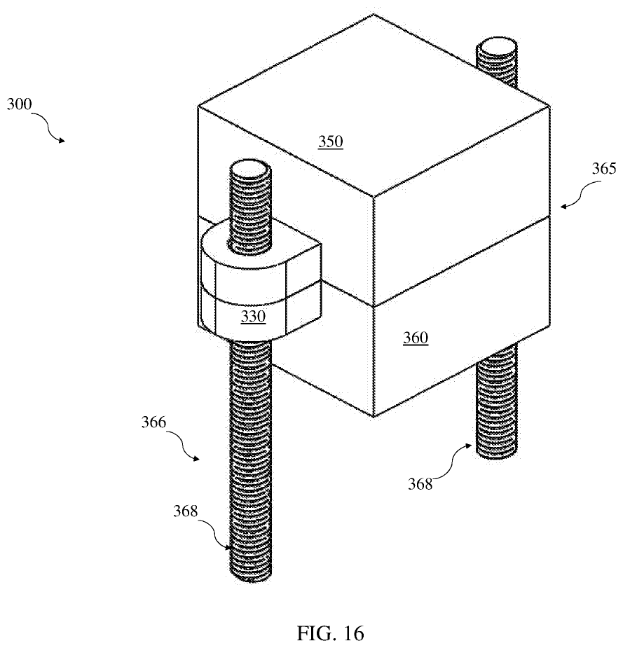

FIG. 13 illustrates a front view, FIG. 14 illustrates a right view, FIG. 15 illustrates a top view, and FIG. 16. illustrates an isometric view of an assembled embodiment of the housing unit 300, including the first section 350 coupled with a complementary second section 360. In certain cases, the first section 350 matingly shapes to the second section 360 such that they contact at an interface 365. As described with reference to first section 350, the second section 360 can also include a base 320 and a set of tabs 330 extending from the base 320. The set of tabs 330 are aligned to enable coupling of the first section 350 with the section 360. In certain cases, the first section 350 and the second section 360 are coupled by a connector 366. In some cases, the connector 366 includes a threaded coupler such as a threaded rod that is sized to fit through and complement the internal threads 340 in each of the tabs 330. In some cases, the connector 366 has external threads 368 for complementing the internal threads 340 in the tabs 330. In the examples depicted in FIGS. 13-18, two connectors 366 are used to connect the first section 350 and the second section 360. It is understood, however, that any number of connectors 366 can be used to connect sections of the housing unit 300, e.g., one or more connectors. In certain cases, the connectors 366 are only able to be actuated (e.g., tightened or loosened) using an external tool that complements the thread spacing and orientation of those threads 368. In some cases, the external tool includes a motorized or non-motorized tool that is configured to spin the connector 366 and cause the housing unit 300 to connect/disconnect.

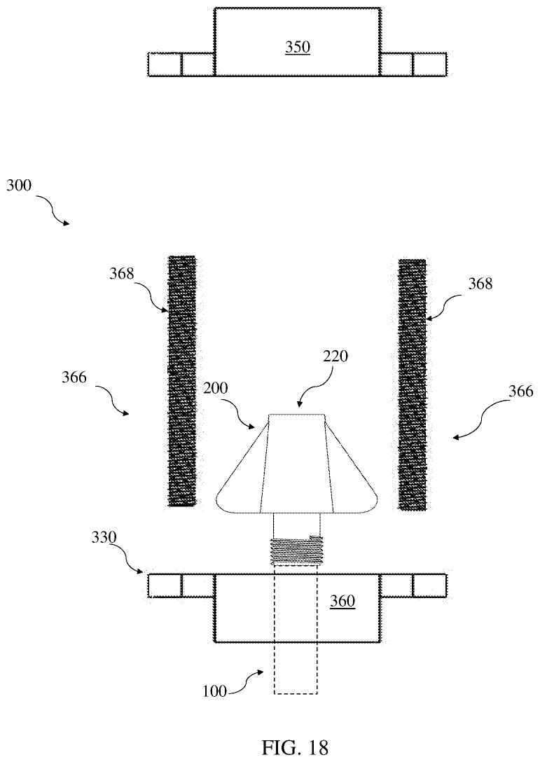

As can be seen in comparing the separated depiction in FIG. 18 with the locked depiction in FIG. 16, when the first section 350 and second section 360 are coupled (e.g., closed and contacting at interface 365), the housing unit 300 encloses the injection port 220. That is, the housing unit 300 is configure to prevent access to the injection port 220 when the first section 350 and the second section 360 are coupled (e.g., contacting or nearly contacting at interface 365). When the housing unit 300 encloses the injection port 220, the injection port 220 is not accessible for insertion or use.

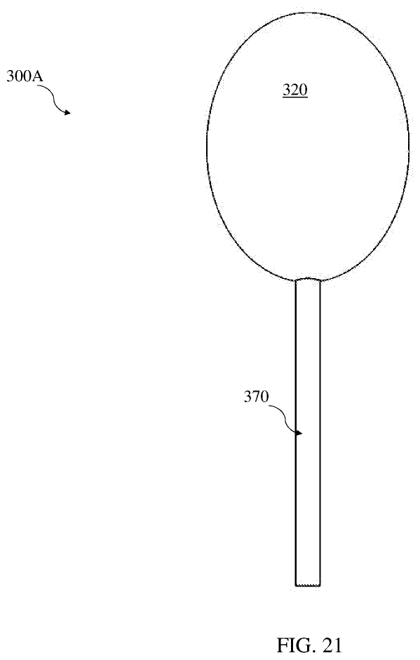

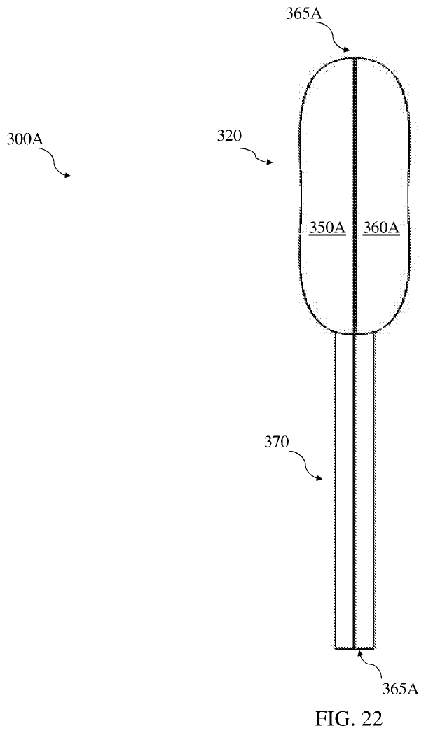

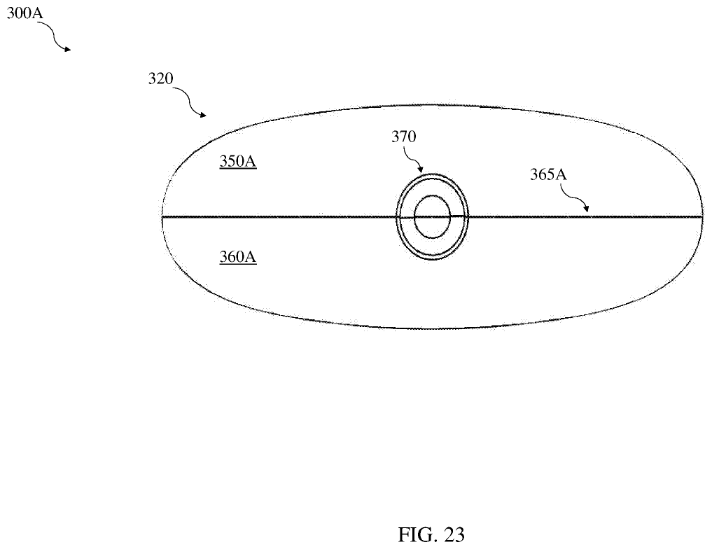

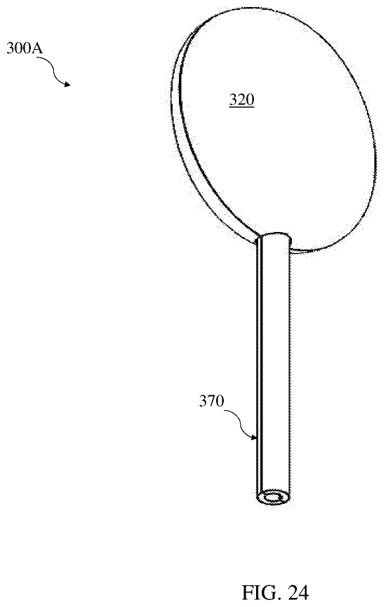

FIGS. 21-24 illustrate additional implementations of a housing unit 300A according to various implementations. In various implementations, the housing unit 300A includes a base 320 and a stem 370 extending from the base 320. In particular, FIG. 21 illustrates a front view, FIG. 22 illustrates a side view, FIG. 23 illustrates a top view, and FIG. 24. illustrates an isometric view, respectively, of the housing unit 300A. In certain cases, the base 320 includes sections 350A, 360A that can be separated to accommodate (e.g., surround) the luer locking mechanism 200. In some cases, the base 320 and the stem 370 are sectioned to allow for accommodating the luer locking mechanism 200. An interface 365A is illustrated between sections 350A, 360A in FIGS. 22 and 23.

In various implementations, the base 320 has an internal slot (not shown) that is sized to receive the luer locking mechanism 200 and at least partially enclose the injection port 220 to prevent access to that injection port 220. In certain cases, the internal slot has a geometry that is configured to at least partially cover the injection port 220. In particular cases, the base 320 completely encloses the luer locking mechanism when the sections 350A, 360A are coupled. In certain cases, the stem 370 is integral with the base 320, and is sized to accommodate the lumen 100. The distal end 160 of the lumen 100 may be partially or fully enclosed by the stem 370 to prevent access to that distal end 160 of the lumen 100. In other cases, the stem 370 is selectively coupleable to the luer locking mechanism 200. In particular cases, sections 350A, 360A of the base 320 completely enclose the luer locking mechanism 200, adding another level of protection against tampering with the injection port 220.

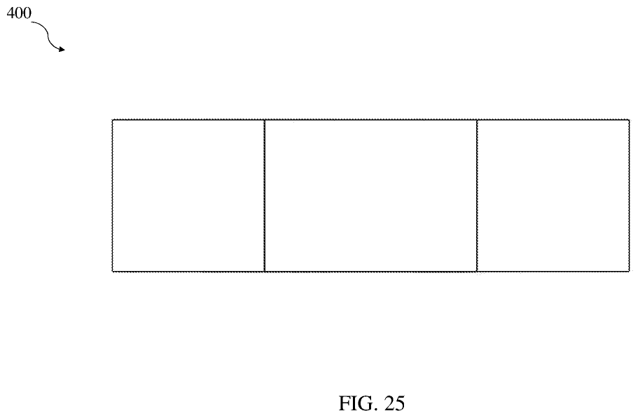

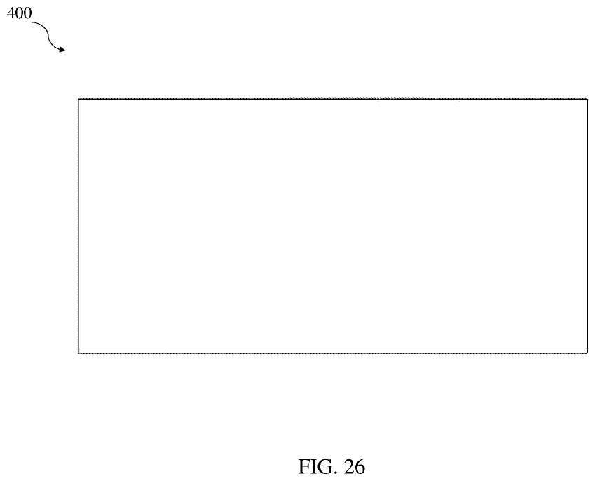

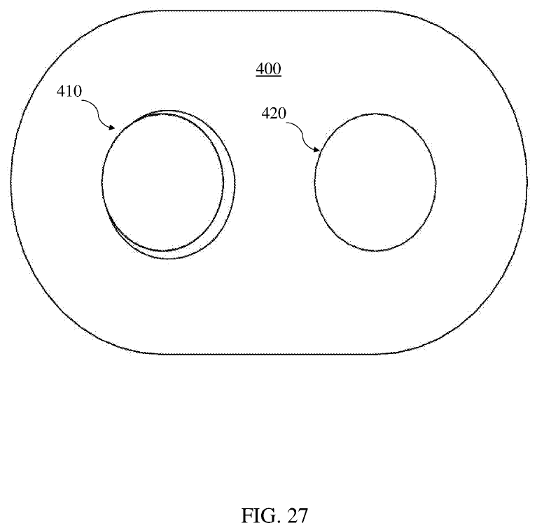

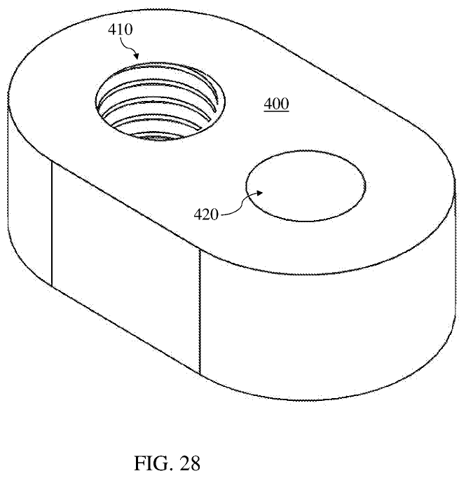

FIGS. 25-28 illustrate different views of a thread pairing 400 for the housing unit 300A according to various implementations. In these cases, the thread pairing 400 includes at least one (internally) threaded hole 410 and one (internally) non-threaded hole 420. In particular implementations, the thread pairing 400 is sized and oriented to keep the first section 350 and the second section 360 of the housing unit 300 coupled.

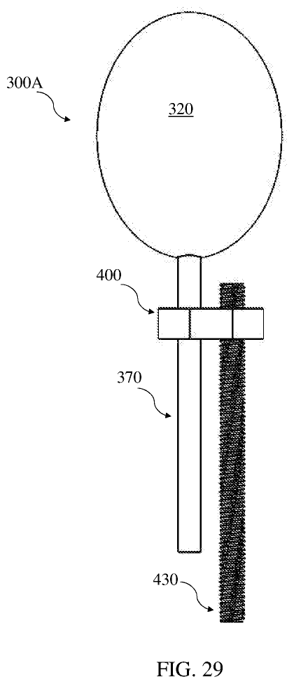

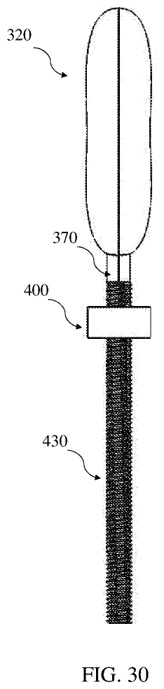

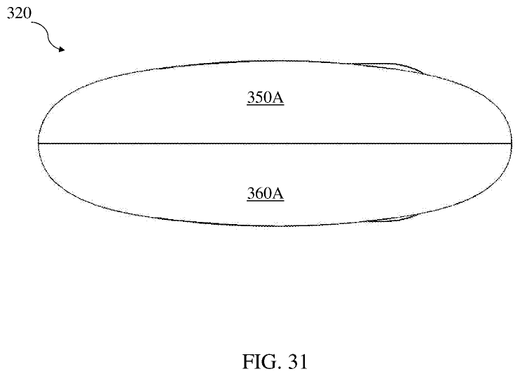

In various implementations, the thread pairing 400 is configured to hold the housing unit 300A together via the stem 370. FIGS. 29-32 illustrate different views of the thread pairing 400 with the housing unit 300A. In the depicted examples, the stem 370 is sized to fit within the non-threaded hole 420 in the thread pairing 400. In other implementations, the stem 370 has external threads and is configured to be inserted into the threaded hole 410. By inserting the stem 370 into the threaded hole 410 or non-threaded hole 420, the first section 350 and second section 360 of the housing unit 300A are held together to enclose the injection port 220. That is, during use as part of a tamper-resistant PICC, the sections 350A, 360A of the housing unit 300A are connected around the luer locking mechanism 200, and the lumen 100 runs through the stem 370 and into the patient. When the thread pairing 400 is in place around the sections 350A, 350B of the housing unit 300A, those sections cannot be separated, preventing access to the injection port 220.

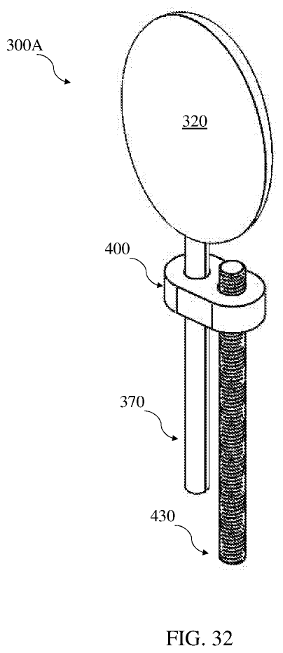

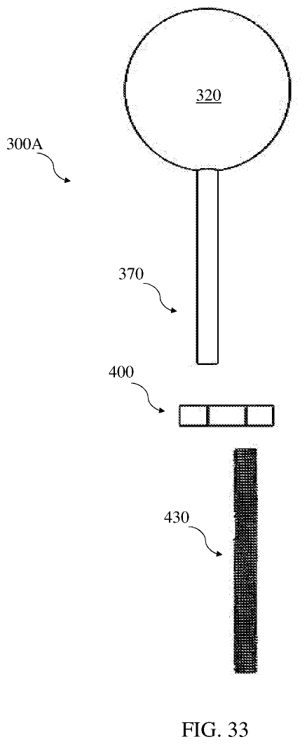

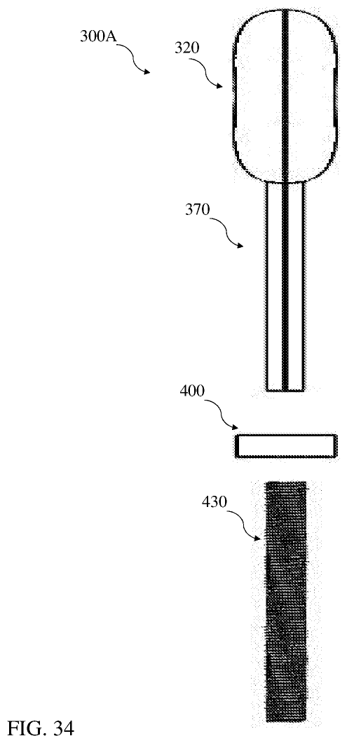

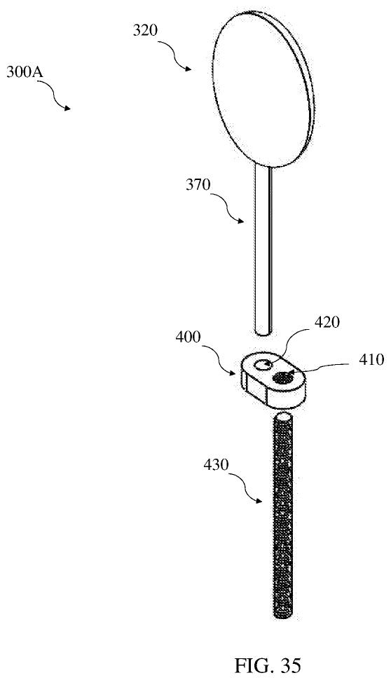

In various implementations, as illustrated in the views in FIGS. 32-35, the thread pairing 400 is actuatable using an external tool 430, e.g., to engage or disengage the thread pairing 400 to the stem 370 of the housing unit 300A. In particular implementations, the external tool 430 includes an externally threaded coupler or rod that is sized to mate with the internal threads in the threaded hole 420. In some cases, the sections of the housing unit 300A are held together by force within the non-threaded hole 420 in the thread paring 400. That is, the size of the non-threaded hole 420 is such the stem 370 cannot move easily within the hole 420. More particularly, the non-threaded hole 420 has an inner dimension that approximates the outer dimension of the stem 370, such that these components are force-fit together and cannot be separated without significant external force (e.g., force greater than the average human's hand can apply). In these cases, in order to remove the stem 370 from the non-threaded hole 420, the external tool 430 is used to rotate the thread pairing 400 about the stem 370. In these cases, the external tool 430 is configured to be rotated such that the thread pairing 400 also rotates around the stem 370 and can be translated (e.g., away from the base 320). In these implementations, as the thread pairing 400 moves farther from the base 320 along the stem 370, the sections 350A, 360A can be separated, allowing access to the luer locking mechanism 200 (and consequently, the injection port 220). Without the external tool 430 (and in some cases, an apparatus to rotate the external tool 430), a user will have difficulty translating the thread pairing 400 in order to access the luer locking mechanism 200.

With reference to several of the FIGURES, e.g., FIGS. 18-20 and 32-35, various additional implementations include a method of inserting and/or removing a PICC from a patient. In one example, the method includes inserting the PICC into a patient. Inserting the PICC can include coupling the luer locking mechanism 200 to the lumen 100. This method can further include positioning the housing unit 300 around the luer locking mechanism 200. In some cases, this can include placing the sections 350, 360 of the housing unit 300 around the luer locking mechanism 200. After positioning the housing unit 300 around the luer locking mechanism 200, the method can further include inserting the proximal end 150 of the lumen 100 into a designated patient area, e.g., into an artery, capillary, vein, vessel, cavity, etc. of the patient. After inserting the lumen 100 into the patient, the method can further include securing the housing unit 300 to the luer locking mechanism 200, e.g., securing the housing unit 300 around the luer locking mechanism 300, to prevent access to the injection port 220.

As noted herein, in various implementations, the housing unit 300 fully encloses the luer locking mechanism 200 to not only prevent access to the injection port 220, but the entire luer locking mechanism 200. In these cases, the luer locking mechanism 200 is visually obstructed by the housing unit 300 as well as being inaccessible. In some cases, the housing unit 300 can only be unlocked using a key, e.g., such as a specifically authorized key. In particular cases, the key is a physical key with teeth or other mating features for engaging a keyhole on the housing unit 300. In other cases, the housing unit 300 can be locked/unlocked using another key mechanism, e.g., a radio frequency identification (RFID) key/card, infra-red (IR) key, visual or facial recognition, keypad, or any other electronic locking mechanism. In some cases, the housing unit 300 can be locked or unlocked via a software application (with corresponding permissions and security provisions) that is executed at least in part on a smart device (e.g., smart phone, smart watch, smart key, etc.) and can be shared with authorized users.

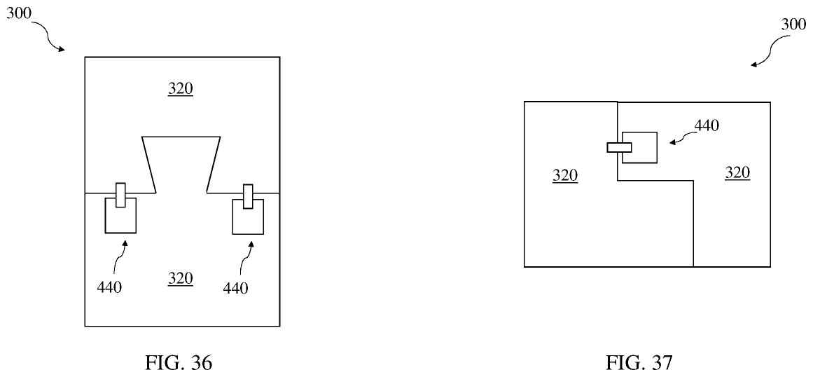

In a particular example, the housing unit 300 is locked/unlocked with a solenoid actuator (e.g., a push-pull mechanism) that is controlled by one or more electronic key mechanisms. In some cases, a solenoid actuator is controlled by an RFID key/card. Example configurations of a housing 300 are illustrated in FIGS. 36 and 37, which each include at least one solenoid actuator 440 for enabling selective locking/unlocking of the sections 320 of the housing 300. In some cases (e.g., FIG. 36), the sections 320 of housing 300 can include one or more dovetailed connections, and at least two solenoid actuators 440 are used to mate these sections 320. In other cases (e.g., FIG. 37), the sections of housing 300 include stepped or recessed (complementary) mating surfaces that can be locked/unlocked using one or more solenoid actuators 440. In certain implementations, the housing 300 can have one or more surface contours for complementing the shape of a user's body, e.g., arm. That is, the surface(s) of the housing 300 can be contoured to mate with the natural bend in a user's forearm.

In some cases, a method includes accessing an inserted PICC in a patient. In these cases, the method can include unlocking the housing unit 300 positioned around the luer locking mechanism 200 to expose the injection port 220. After unlocking the housing unit 300, the method further includes inserting a medical device (e.g., syringe) into the injection port 220, e.g., a medical device with an injection angle that complements the specific injection angle of that port 220. After inserting (or otherwise coupling) the syringe with the injection port 220, the method includes injecting a fluid into the patient via the lumen 100.

In additional implementations, a method includes removing an inserted PICC from a patient. In various implementations, the method of removing the PICC can include: unlocking the housing unit 300 to expose the luer locking mechanism 200. After unlocking the housing unit 300, the sections of the housing unit 350, 360 can be removed from around the luer locking mechanism 200. In some cases, after unlocking the housing unit 300, the coupled luer locking mechanism 200 and lumen 100 are removed from the patient in one process. In other cases, the entire PICC is removed from the patient without unlocking the housing unit 300. In any case, removing the inserted PICC can be performed to cause the least amount of discomfort, damage and/or injury to the patient as possible.

It is understood that the processes of inserting, accessing and/or removing the PICC can be performed by the same person and/or entity (e.g., doctor and/or hospital). However, in other cases, the PICC is inserted, accessed and removed by one or more distinct persons or entities.

In order to clearly describe the current technology it may be necessary to select certain terminology when referring to and describing relevant components within PICCs (or PICC lines). To the extent possible, common industry terminology is used and employed in a manner consistent with its accepted meaning. Unless otherwise stated, such terminology should be given a broad interpretation consistent with the context of the present application and the scope of the appended claims. Those of ordinary skill in the art will appreciate that often a particular component may be referred to using several different or overlapping terms. What may be described herein as being a single part may include and be referenced in another context as consisting of multiple components. Alternatively, what may be described herein as including multiple components may be referred to elsewhere as a single part.

It is often required to describe parts that are disposed at differing radial positions with regard to a center axis. The term “radial” refers to movement or position perpendicular to an axis. For example, if a first component resides closer to the axis than a second component, it will be stated herein that the first component is “radially inward” or “inboard” of the second component. If, on the other hand, the first component resides further from the axis than the second component, it may be stated herein that the first component is “radially outward” or “outboard” of the second component. The term “axial” refers to movement or position parallel to an axis. Finally, the term “circumferential” refers to movement or position around an axis. It will be appreciated that such terms may be applied in relation to the center axis of the device.

In addition, several descriptive terms may be used regularly herein, as described below. The terms “first”, “second”, and “third” may be used interchangeably to distinguish one component from another and are not intended to signify location or importance of the individual components.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components but do not preclude the presence or addition of one or more other features, integers, steps, operations, elements, components, and/or groups thereof. “Optional” or “optionally” means that the subsequently described event or circumstance may or may not occur, and that the description includes instances where the event occurs and instances where it does not.

Where an element or layer is referred to as being “on,” “engaged to,” “connected to” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

One or more components described herein can be formed according to known manufacturing methods, e.g., molding, casting, forging or additive (e.g., three-dimensional) manufacturing, and can be formed from known materials, e.g., a metal such as aluminum or steel, a thermoplastic material (e.g., polycarbonate (PC) or acrylonitrile butadiene styrene (ABS)) or a composite material (e.g., PC/ABS). Certain components can include materials used for damping motion, such as silicone, a thermoplastic (e.g., POM) or a thermoplastic elastomer (TPE).

In various implementations, components described as being “coupled” to one another can be joined along one or more interfaces. In some implementations, these interfaces can include junctions between distinct components, and in other cases, these interfaces can include a solidly and/or integrally formed interconnection. That is, in some cases, components that are “coupled” to one another can be simultaneously formed to define a single continuous member. However, in other implementations, these coupled components can be formed as separate members and be subsequently joined through known processes (e.g., soldering, fastening, ultrasonic welding, bonding).

A number of implementations have been described. Nevertheless, it will be understood that additional modifications may be made without departing from the scope of the inventive concepts described herein, and, accordingly, other implementations are within the scope of the following claims.

We claim:

1. A peripherally inserted central catheter (PICC), comprising:
a lumen having: an innermost layer, at least one middle layer, an outermost layer, and a proximal end for inserting into a vessel, wherein the lumen is puncture resistant;
a luer locking mechanism coupled to a distal end of the lumen, the luer locking mechanism having at least one injection port for mating with an external injectable medical device; and
a housing unit at least partially surrounding the luer locking mechanism and preventing access to the at least one injection port.

2. The PICC of claim 1, wherein:
the innermost layer includes at least one of polyurethane or silicone;
the middle layer includes at least one of a metal, a polymer, a composite, or a synthetic fiber; and
the outer layer includes at least one of polyurethane or silicone.

3. The PICC of claim 1, wherein the luer locking mechanism has a coupler for matingly engaging a coupler at the distal end of the lumen.

4. The PICC of claim 3, wherein the luer locking mechanism engages with the distal end of the lumen via a set of interlocking threads, and wherein the set of interlocking threads have a length and a pitch that deviates from a reference thread by approximately ten percent.

5. The PICC of claim 1, wherein the luer locking mechanism has an entrance angle that deviates from a reference injection angle for a medical device port by at least approximately ten percent.

6. The PICC of claim 1, wherein the housing unit is selectively coupleable to an actuator for at least one of opening or closing the housing unit around the luer locking mechanism.

7. The PICC of claim 1, wherein the housing unit includes a first section and a second section, wherein the first section and the second section are matingly shaped, and wherein when the first section is coupled with the second section the housing unit encloses the injection port.

8. The PICC of claim 7, wherein when the first section is coupled with the second section, the housing unit prevents access to the injection port.

9. The PICC of claim 7, wherein the housing unit has a means for selectively coupling the first section with the second section, and wherein the selective coupling is performed with an external tool.

10. The PICC of claim 1, wherein the housing unit has a slot that is sized to receive the luer locking mechanism.

11. The PICC of claim 1, wherein the housing unit has a base and a set of tabs extending from the base, wherein the tabs include a set of interlocking threads, and wherein the set of interlocking threads have a length and a pitch that deviate from a reference thread by approximately ten percent.

12. The PICC of claim 1, wherein the housing unit comprises a base and a stem, wherein the stem is coupleable to the luer locking mechanism.

13. The PICC of claim 12, further comprising: a thread pairing having at least one threaded hole and at least one non-threaded hole, wherein in a held position the stem is retained in the non-threaded hole.

14. The PICC of claim 13, wherein the threaded hole of the thread pairing is selectively coupleable to an external tool for at least one of engaging or disengaging the stem of the housing unit.

15. A method comprising:
inserting a peripherally inserted central catheter (PICC) into a patient, the inserting including:
coupling a luer locking mechanism to a lumen, the luer locking mechanism having an injection port;
coupling a housing unit to the luer locking mechanism;
inserting the lumen into a designated patient area; and
securing the housing unit to the luer locking mechanism to inhibit access to the injection port.

16. The method of claim 15, wherein securing the housing unit to the luer locking mechanism comprises using an authorized key to lock the housing unit.

17. The method of claim 16, further comprising unlocking the housing unit using the authorized key.

18. The method of claim 15, wherein the inserting further comprises coupling a thread pairing to the housing unit to secure the housing unit around the luer locking mechanism.

19. The method of claim 18, wherein the inserting further comprises: using the thread pairing to inhibit access to the injection port by securing the housing unit around the luer locking mechanism.

20. The method of claim 15, further comprising removing the inserted PICC from the patient, the removing comprising:
disengaging the luer locking mechanism from the patient; and
guiding the lumen out of the patient.
